(19) United States
(12) Patent Application Publication
(10) Pub. No.: US 2019/0219693 A1
(43) Pub. Date: Jul. 18, 2019

(54) 3-D US Volume From 2-D Images From Freehand Rotation and/or Translation of Ultrasound Probe

(71) Applicant: BK MEDICAL HOLDING COMPANY, INC., Peabody, MA (US)

(72) Inventors: David LIEBLICH, Worcester, MA (US); Spiros MANTZAVINOS, Lynn, MA (US)

(73) Assignee: BK Medical Holding Company, Inc., Peabody, MA (US)

(21) Appl. No.: 16/301,652

(22) PCT Filed: May 16, 2016
(86) PCT No.: PCT/US2016/032639

§ 371 Date: Nov. 14, 2018

(51) Int. Cl. G01S 15/89; A61B 8/12; A61B 8/00; A61B 8/08

(52) U.S. Cl. CPC G01S 15/8993; A61B 8/12; G01S 15/8936; A61B 8/4483; A61B 8/483; A61B 8/4444

(57) ABSTRACT

A method includes free hand rotating or translating a first transducer array by rotating or translating the probe (102) about or along a longitudinal axis (206) of the probe through a plurality of angles or linear displacements in a cavity, moving a first imaging plane through an extent of a structure of interest. The method further includes transmitting signals and receiving echoes with the first transducer array concurrently with the rotating or the translating, and generating two-dimensional images of the structure of interest with the received echo for the plurality of the angles or the linear displacements. The method further includes identifying the plurality of the angles or the linear displacements based on the generated images and secondary information, aligning the two-dimensional images based on the identified plurality of the angles or the linear displacements, and combining the aligned two-dimensional images to construct a three-dimensional volume of the structure of interest.

### TECHNICAL FIELD

The following generally relates to ultrasound imaging and more particularly to constructing a three-dimensional (3-D) ultrasound volume from two-dimensional (2-D) ultrasound images acquired during freehand rotation and/or translation of an ultrasound probe.

### BACKGROUND

An ultrasound imaging system has included a probe with transducer array that transmits an ultrasound beam into an examination field of view. As the beam traverses structure (e.g., in an object or subject) in the field of view, sub-portions of the beam are differentially attenuated, scattered, and/or reflected off the structure, with some of the energy reflected back towards the transducer array. The transducer array receives the echoes, which are processed to generate one or more images of the structure.

During fusion biopsy, a real-time two-dimensional (2-D) ultrasound image is fused with a previously acquired 3-D volume, to locate targets (potential lesions) previously identified within the 3D volume. The current position of a transducer probe is tracked with respect to the scanned anatomy, and navigated to a target based upon the current location relative to that of the previously identified biopsy target. The 3-D volume has been an MRI, CT, etc. volume.

Unfortunately, such approaches have required additional instrumentation to track position and orientation of the ultrasound probe, for example: magnetic, electromagnetic, optical, and/or acoustic sensors, etc. These sensors add cost and complexity, and increase setup/breakdown time and system footprint. An alternative approach relies on the extraction of positioning information directly from the real-time 2-D ultrasound image using speckle correlation. However, this approach generally is computationally intensive, time consuming, and less reliable than using position and orientation sensors.

### SUMMARY

Aspects of the application address the above matters, and others.

In one aspect, a method includes free hand rotating or translating a first transducer array of a probe by rotating or translating the probe about or along a longitudinal axis of the probe through a plurality of angles or linear displacements in a cavity, wherein the rotating or the translating moves a first imaging plane of the first transducer array through an extent of a structure of interest. The method further includes transmitting ultrasound signals and receiving echo signals with the first transducer array concurrently with the rotating or the translating the first transducer array. The method further includes generating spatially sequential two-dimensional images of the structure of interest with the received echo signals for the plurality of the angles or the linear displacements. The method further includes identifying the plurality of the angles or the linear displacements based on the generated images and secondary information. The method further includes aligning the two-dimensional images based on the identified plurality of the angles or the linear displacements. The method further includes combining the aligned two-dimensional images to construct a three-dimensional volume including at least the structure of interest.

In another aspect, an ultrasound probe includes at least one transducer array configured to transmit and receive echoes and a three-dimensional processor. The three-dimensional processor is configured to align a set of image planes generated from the echoes for different rotation angles of the at least one transducer array or different displacements of the at least one transducer array based on a signal indicative of the different rotation angles or the different displacements. The three-dimensional processor is further configured to combine the aligned image planes to construct volumetric ultrasound image data of a structure of interest.

In another aspect, a non-transitory computer readable medium is encoded with computer executable instructions, which, when executed by a computer processor, causes the processor to: acquire image planes with a rotating or translating first transducer array of a rotating or translating probe, determine rotation angles or displacements for the image planes based on one of an image of a transverse plane or a signal from a motion sensor of the probe, wherein each image plane includes a different sub-portion of a structure of interest, align the image planes based on the determined rotation angles or displacements, and construct a three-dimensional data set of the structure of interest with the aligned image planes.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example ultrasound imaging system configured to generate a 3-D ultrasound volume from 2-D ultrasound images captured by freehand rotation and/or translation of the probe;

FIG. 2 schematically illustrates a side view of a biplane probe;

FIG. 3 schematically illustrates a perspective view of the biplane probe;

FIG. 4 schematically illustrates a perspective view of an end-fire probe with a motion sensor;

FIG. 5 schematically illustrates a side view of a sagittal plane probe with a motion sensor;

FIG. 6 schematically illustrates a perspective view of an axial plane probe with a motion sensor;

FIG. 7 illustrates an example method employing a biplane probe to generate a 3-D ultrasound volume with 2-D ultrasound images captured with freehand rotation or translation of the probe;

FIGS. 8, 9 and 10 respectively show an example of the progressive rotation of a structure of interest in axial planes for three axial rotation angles;

FIGS. 11, 12 and 13 respectively show corresponding sagittal planes of the structure of interest for the three axial rotation angles;

FIG. 14 graphically shows the shift of the of the structure of interest in the images of FIGS. 8, 9 and 10 as a function of rotation angle;

FIG. 15 graphically shows the two left most rotations as translations parallel to radial coordinate lines;

FIG. 16 illustrates an example method employing an end-fire or sagittal array to generate a 3-D ultrasound volume with 2-D ultrasound images captured with freehand rotation of the probe; and

FIG. 17 illustrates an example method employing an axial array to generate a 3-D ultrasound volume with 2-D ultrasound images captured with freehand translation of the probe.

### DETAILED DESCRIPTION

The following describes an approach for constructing a 3-D ultrasound volume from 2-D ultrasound images acquired through freehand rotation about and/or freehand translation along a longitudinal axis of an ultrasound probe, along with at least one of an axial image, a sagittal image, and, rotation or displacement information from a sensor on the probe. Alternatively an axial and sagittal image is sufficient.

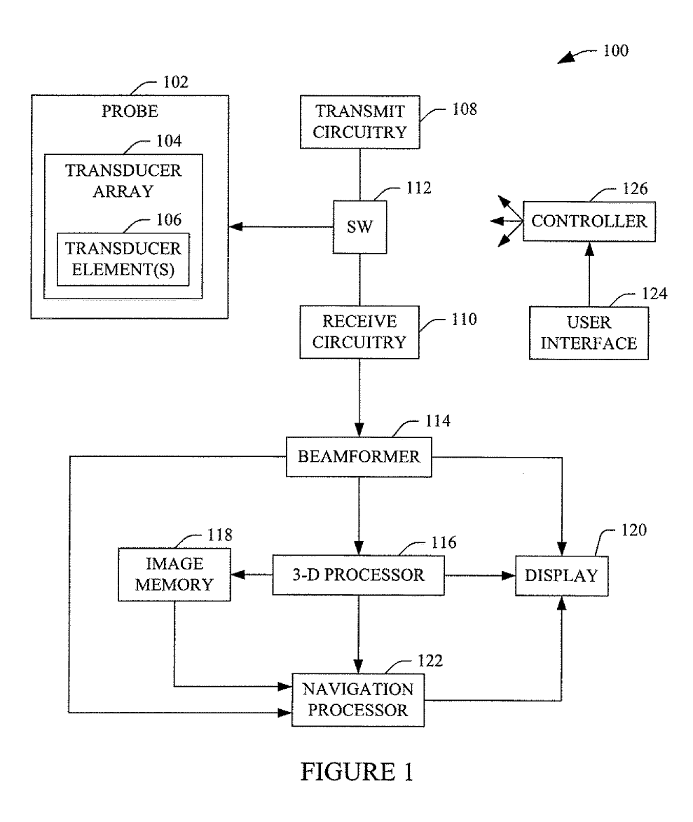

Initially referring to FIG. 1, an ultrasound imaging system 100 includes a probe 102 housing a transducer array 104 having at least one transducer element 106. The at least one transducer element 106 is configured to convert electrical signals to an ultrasound pressured field and vice versa respectively to transmit ultrasound signals into a field of view and receive echo signals, generated in response to interaction with structure in the field of view. The illustrated transducer array 104 can include one or more arrays, including linear, curved (e.g., concave, convex, etc.), circular, area etc. arrays, which are fully populated or sparse, etc.

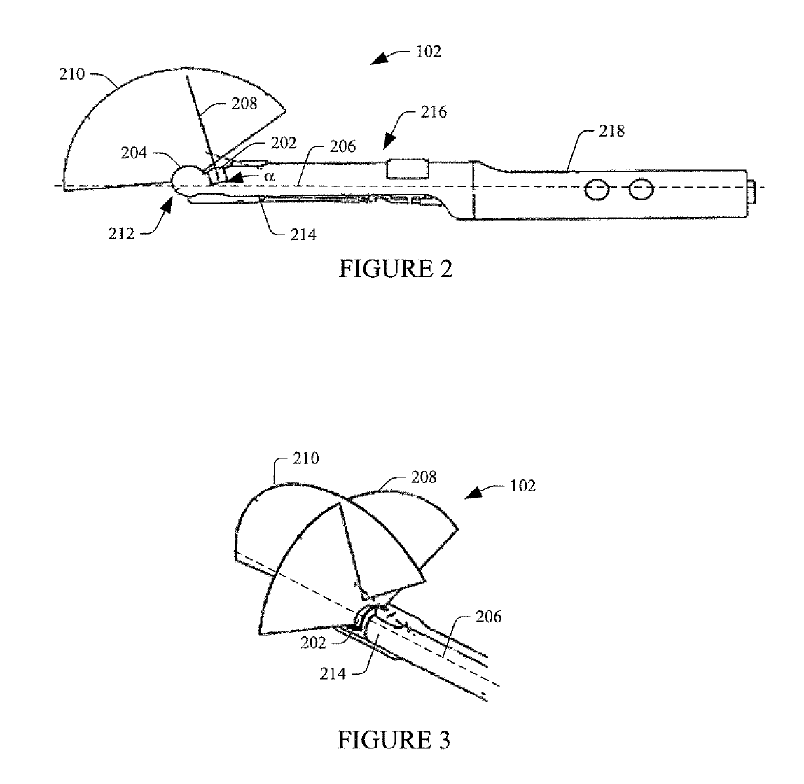

FIGS. 2 and 3 schematically illustrate an embodiment in which the probe 102 is a biplane probe where the transducer array 104 includes an axial array 202 and a sagittal array 204, which are transverse to each other with respect to a longitudinal axis 206 and have corresponding image planes 208 and 210 that intersect. Generally, the image planes 208 and 210 are known planes with respect to the axis of rotation and do not have to intersect. In the illustrated embodiment, the array 202 is disposed at an incline of α=15° with respect to the longitudinal axis 206. Suitable angles include an angle in a range of α=0° (parallel to the longitudinal axis 206) to α=90°. For example, for the prostate, having an axial plane view (or something close to it) is desirable as urologists are familiar with structures in this plane and the sagittal plane, and sometimes, the prostate is more clearly discernable in one than the other. Generally, a suitable angular range depends on the application. The arrays 202 and 204 are located at a tip or first end region 212 of a shaft 214, with an opposing or second end region 216 of the shaft 214 coupled to a handle 218.

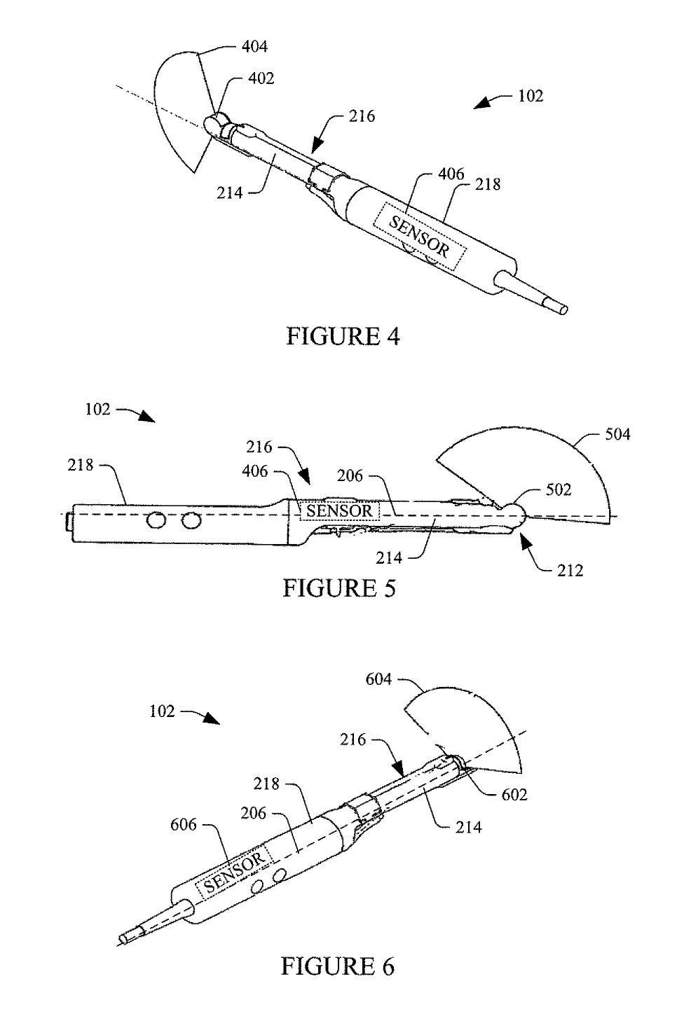

FIG. 4 schematically illustrates an embodiment of the probe 102 in which the transducer array 104 includes an end-fire array 402 having a single image plane 404. With this embodiment, the probe 102 includes at least one sensor 406 with at least one degree of freedom that senses rotations about the longitudinal axis 206. The illustrated location of the sensor 406 is not limiting, and the sensor 406 can be located anywhere on the probe 102 where it can sense the rotations about the longitudinal axis 206, including inside and/or exterior to the handle 218 and/or the shaft 214. Examples of suitable sensors include single or multi-axis gyroscopes and accelerometers, etc.

FIG. 5 schematically illustrates an embodiment in which the transducer array 104 has only a sagittal array 502 with a sagittal plane 504 (or only the sagittal array 204 of the configuration of FIGS. 2 and 3 is employed). This configuration, similar to FIG. 4, includes the sensor 406 with the at least one degree of freedom that senses rotations about the longitudinal axis 206. FIG. 6 schematically illustrates an embodiment in which the transducer array 104 has only an axial array 602 with an axial plane (or only the axial array 202 of the configuration of FIGS. 2 and 3 is employed). This configuration includes a sensor 606 with at least one degree of freedom that senses displacements along the longitudinal axis 206 for an axial plane probe.

Returning to FIG. 1, transmit circuitry 108 generates a set of pulses (or a pulsed signal) that are conveyed, via hardwire (e.g., through a cable) and/or wirelessly, to the transducer array 104. The set of pulses excites a set (i.e., a sub-set or all) of the at least one transducer element 106 to transmit ultrasound signals. Receive circuitry 110 receives a set of echoes (or echo signals) generated in response to a transmitted ultrasound signal interacting with structure in the field of view. A switch (SW) 112 controls whether the transmit circuitry 108 or the receive circuitry 110 is in electrical communication with the at least one transducer element 106 to transmit ultrasound signals or receive echoes.

A beamformer 114 processes the received echoes by applying time delays to echoes, weighting echoes, summing delayed and weighted echoes, and/or otherwise beamforming received echoes, creating beamformed data. In B-mode imaging, the beamformer 114 produces a sequence of focused, coherent echo samples along focused scanlines of a scanplane. The scanplanes correspond to the axial and/or sagittal planes of the transducer array 104. The beamformer 114 may also process the scanlines to lower speckle and/or improve specular reflector delineation via spatial compounding, and/or perform other processing such as FIR filtering, IIR filtering, edge enhancement, etc.

A three-dimensional processor 116 is configured to process the scanplanes and generate a 3-D volume. As described in greater detail below, in one instance this includes processing 2-D images for two different (e.g., transverse) image planes acquired with two different arrays, one plane being rotated about the axis 206 to capture three-dimensional image data of structure of interest using the other plane as a frame of reference and/or guide. Another approach includes processing images for a single plane acquired with a single array, which is rotated about or translated along the axis 206 to capture three-dimensional data of structure of interest, while using rotation or displacement information from a sensor of the probe rotating or translating with the probe as a frame of reference and/or guide.

The resulting 3-D volume can be stored in image memory 118, memory external to the system 100, visually displayed via a display monitor 120, employed to facilitate real-time navigation in conjunction with real-time 2-D ultrasound images, etc. For the latter, a navigation processor 122 registers real-time 2-D ultrasound images with the 3-D volume. This information can be used to identify the location and/or orientation of the ultrasound transducer 104 relative to the current location of the scanned anatomy, and move the ultrasound transducer 104 to the structure of interest. The 3-D volume can be rendered with the real-time 2-D ultrasound image superimposed thereover and/or with graphical indicia indicating information such as the transducer, instrument and/or structure location. In a variation, the navigation processor 122 is omitted or separate from the system 100.

The approach described herein reduces the cost and complexity of the system as well as setup/breakdown time and system footprint, as compared to an external navigation systems and reduces processing time compared to a speckle-based approach. Furthermore, at least the example with the biplane probe does not require any additional motion sensing components and thus mitigates this additional cost and the complexity of modifying the system to use the information therefrom. Moreover, employing the ultrasound 3-D volume rather than directly positioning or extracting positioning information from the real-time 2-D image may result in improved accuracy.

A user interface (UI) 124 includes an input device(s) (e.g., a physical button, a touch screen, etc.) and/or an output device(s) (e.g., a touch screen, a display, etc.), which allow for interaction between a user and the ultrasound imaging system 100. A controller 126 controls one or more of the components 102-124 of the ultrasound imaging system 100. Such control includes controlling one or more of the components to perform the functions described herein and/or other functions.

In the illustrated example, at least one of the components of the system 100 (e.g., the component) can be implemented via one or more computer processors (e.g., a microprocessor, a control processing unit, a controller, etc.) executing one or more computer readable instructions encoded or embodied on computer readable storage medium (which excludes transitory medium), such as physical computer memory, which causes the one or more computer processors to carry out the various acts and/or other functions and/or acts described herein. Additionally or alternatively, the one or more computer processors can execute instructions carried by transitory medium such as a signal or carrier wave.

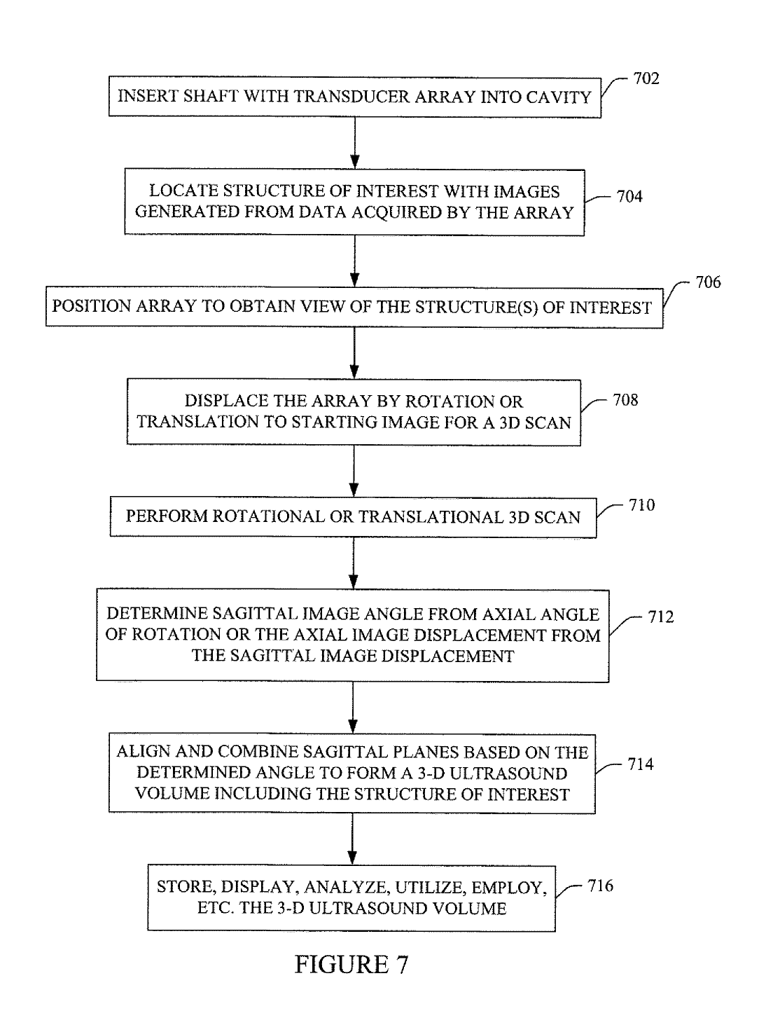

FIG. 7 illustrates an example method for generating a 3-D ultrasound volume using 2-D ultrasound images captured with freehand rotation of the probe 102. For this example, the probe 102 is configured with the biplane transducer array configuration of FIGS. 2 and 3.

It is to be appreciated that the ordering of the above acts is not limiting. As such, other orderings are contemplated herein. In addition, one or more acts may be omitted and/or one or more additional acts may be included.

At 702, the bi-plane ultrasound probe 102 is inserted into a cavity. For example, for a prostate examination, the end 212 of the shaft 214 with the axial and sagittal arrays 202 and 204 is inserted into the rectum.

At 704, the transducer array 104 is used to locate structure of interest in the cavity. This can be achieved, e.g., by activating at least one of the axial transducer array 202 or the sagittal transducer array 204 to image during insertion and using the generated images to locate the structure of interest. This may also include locating, via the images, known other structure in the scan field of view to facilitate locating the structure of interest. With the prostate examination, this may include locating the prostate alone or the prostate and the bladder, the pubic symphysis, etc. in the images.

At 706, the transducer array 104 is positioned using the images from that array to obtain a full field of view of the structure(s) of interest.

At 708, the transducer is displaced by rotation or translation from this image to the location for the starting image of the 3D scan. For example, for a sagittal plane rotational scan, this may be the right or left edge of the prostate, as viewed in the axial plane; in an axial translational scan this may be the apex or base of the prostate as seen in the sagittal plane.

At 710, a rotational or translational 3D scan is performed by acquiring ultrasound planes rotated, via freehand, angularly about the probe axis or translated, via freehand, linearly along the axis direction.

In one instance, to obtain a finely sampled volume and reduce interpolation of image data between 2-D images, the rate of rotation of the probe about its axis is maintained substantially constant while the update frequency for the images produced by the transducer array 104 is fixed and typically in the range of thirty to 100 Hertz (30-100 Hz). For example, a sampling as dense as one plane per degree would require an approximately constant rate of rotation of thirty degrees per second for a duration of three seconds, if the image update frequency is 30 Hz. A visual and/or audible guide can be provided to indicate the appropriate rate of rotation and/or when it is exceeded. In this example, the rotation is performed freehand by a clinician. For embodiments in which the probe 102 is translated, the guide can provide the appropriate rate of translation and/or when it is exceeded. Freehand rotation and/or translation can be accomplished with sufficient precision and minimal training with the probe 102 described herein.

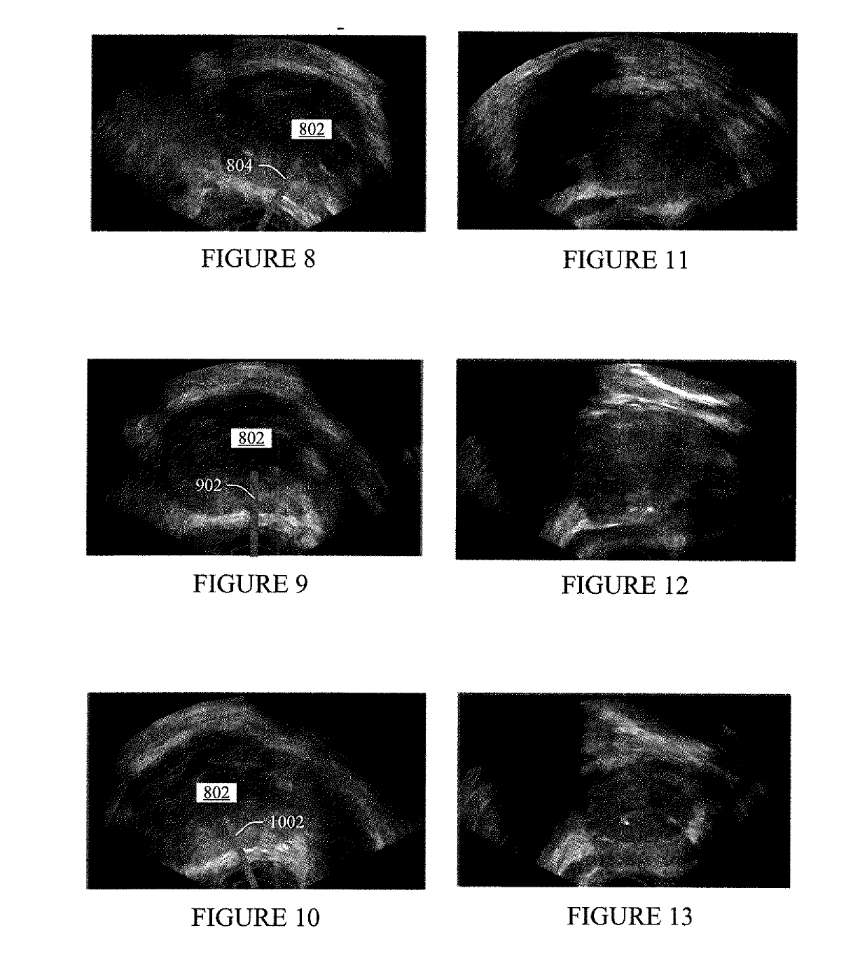

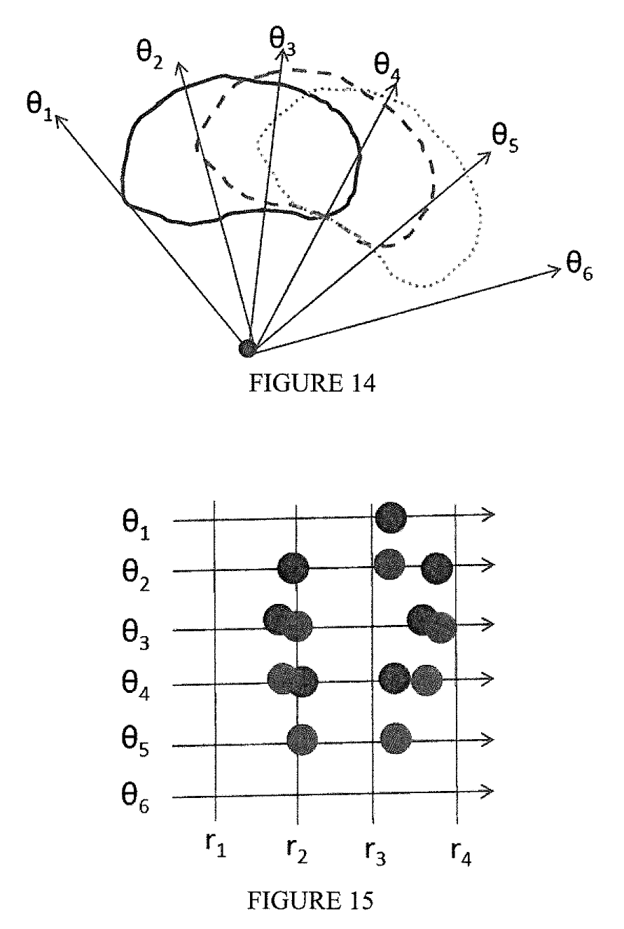

At 712, the axial rotation angle at which a sagittal image is acquired is used to determine an angle for the sagittal image and/or the sagittal image displacement is used of determine the axial image displacement. FIGS. 8, 9 and 10 respectively show an example of the progressive rotation of the axial planes for three example angles θ₁, θ₂ and θ₃. The probe 102 rotation can be detected, e.g., by a shift in position of common identifiable regions between two images. The shift can be seen in the axial images of FIGS. 8, 9 and 10 with the shift of a structure 802 respectively from 804, through 902, to 1002. FIGS. 11, 12 and 13 respectively show corresponding sagittal planes for the angles θ₁, θ₂ and θ₃. FIG. 14 graphically shows the shift of the structure 802 in the images of FIGS. 8, 9 and 10 as a function of probe rotation angle θ. FIG. 15 shows the rotations of FIG. 14 as translations parallel to radial coordinate lines rᵢ. Non-rotational motion in the plane will move the points off their corresponding r line. As such, the representation of FIG. 15 can be used to check the quality of the sweep based on the amount of measurable offset from a radial coordinate line in the plane.

The angle for any particular sagittal plane can be based on a single position (e.g., the start angle), relative to any prior position, both, and/or otherwise. The difference between finding angles relative to a single position and relative to a prior position is the difference in potential accumulated angular errors and in the potential requirement to correct for view angle differences in the former case, when the axial plane is not perpendicular to the probe axis. Generally, small rotations, e.g., from adjacent samples in θ, successively detected, can accumulate error but are consistently measurable throughout the angular range of the scan, whereas large rotations, e.g., relative to a single starting plane (e.g., θ₁) do no accumulate multiple errors but are may not be measurable at large angle offsets when the starting plane may no longer be within the field of view. Additionally, measurements relative to a single plane may require view correction, which is negligible for small angles, if the axial plane is not orthogonal to the rotation axis 206 (e.g., FIGS. 2 and 3). A correction for detected angle between images (Δθ) and inclination (α) of the view plane from orthogonal to the axis of rotation is:

$$Δ {θ}^{′} = Acos ( \frac{cos ( Δ θ )}{cos ( {α}^{2} )} - tan ( {α}^{2} ) ) .$$

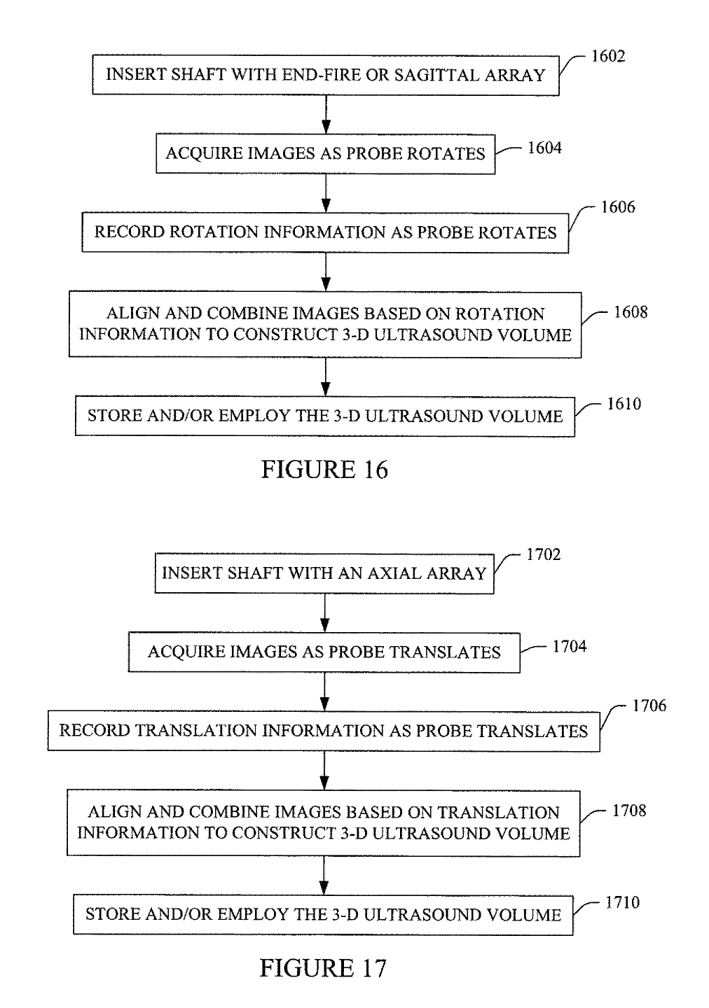

At 714, the sagittal images are aligned and combined to create a 3-D ultrasound volume containing at least the structure of interest. In one instance, this includes aligning the sagittal images at their correct angular position relative to the axis of rotation determined from the known details of the sagittal plane image relative to the axis 206 of the ultrasound probe. For the end-fire configuration of FIG. 4 and/or the sagittal plane configuration of FIG. 5, individual images corresponding to the different planes are aligned and combined to form the 3-D volume based on a value and/or signal from the at least one sensor 406 that senses rotations about the longitudinal axis 206 for a 3-D sweep. For the axial plane configuration of FIG. 6, individual images corresponding to the different axial planes are aligned and combined to form the 3-D volume based on a value and/or signal from the at least one sensor 406 that senses displacements along the axis 206 for an axial plane probe for a linear 3-D scan. End-fire, sagittal plane and axial plane configurations are further discussed in FIGS. 16 and 17.

At 716, the 3-D ultrasound volume is stored, displayed, analyzed, utilized to show previously determined information, employed for an image guided procedure, and/or otherwise used. For example, in one instance, the 3-D ultrasound volume is analyzed to detect tissue of interest such as an organ of interest (e.g., the prostate), lesions, tumors, etc. The resulting 3-D ultrasound volume can be used instead of a previously acquired and analyzed MRI, CT, etc. volumetric image data set for an image guided procedure.

In another instance, structure of interest (e.g., a tumor) identified in a previous 3-D volumetric data from an MRI, CT, etc. scan can be transferred to the 3-D ultrasound volume for the image guided procedure. For example, the 3-D ultrasound volume (e.g., boundaries of structures) can be deformably registered with the previous 3-D volumetric data (e.g., boundaries of structures) and structure of interest identified therein can be mapped or transferred to the 3-D ultrasound volume. The 3-D ultrasound volume with the identified structure can be further analyzed to further add and/or remove structure of interest. The 3-D ultrasound volume can then be used during a procedure in which a 2-D real-time ultrasound image is registered to the 3-D ultrasound volume to determine a location and/or orientation of the transducer array with respect to the anatomy in the 3-D ultrasound volume, including the structure of interest, and navigate the transducer array to the structure of interest, e.g., for a biopsy, to implant a radioactive seed, etc.

FIG. 16 illustrates another example method for generating a 3-D ultrasound volume using 2-D images captured with freehand rotation of the probe 102. For this example, the probe 102 is configured with the end-fire or sagittal array configuration in FIGS. 4 and 5.

It is to be appreciated that the ordering of the above acts is not limiting. As such, other orderings are contemplated herein. In addition, one or more acts may be omitted and/or one or more additional acts may be included.

At 1602, the ultrasound probe 102 is inserted into a cavity to a location of interest, as described herein and/or otherwise.

At 1604, a set of images are acquired as the probe 102 is freehand rotated through an arc about the longitudinal axis 206.

At 1606, concurrently with act 1604, rotation information is generated by the sensor 406 and recorded as the probe 102 rotates.

At 1608, the 2-D ultrasound images are aligned and combined based on the information from the sensor 406 to construct the 3-D ultrasound volume containing the structure of interest.

At 1610, the 3-D ultrasound volume is stored and/or employed (e.g., displayed, analyzed, utilized to show previously determined information, employ for an image guided procedure, etc.) as described herein and/or otherwise.

FIG. 17 illustrates an example method for generating a 3-D ultrasound volume using 2-D images captured with freehand translation of the probe 102. For this example, the probe 102 is configured with an axial array configuration such as the configuration illustrated in FIG. 6.

It is to be appreciated that the ordering of the above acts is not limiting. As such, other orderings are contemplated herein. In addition, one or more acts may be omitted and/or one or more additional acts may be included.

At 1702, the ultrasound probe 102 is inserted into a cavity to a location of interest, as described herein and/or otherwise.

At 1704, a set of images are acquired as the probe 102 is freehand translated along the longitudinal axis 206.

At 1706, concurrently with act 1704, displacement information is generated by the sensor 606 and recorded as the probe 102 translates.

At 1708, the 2-D ultrasound images are aligned and combined based on the information from the sensor 606 to construct the 3-D ultrasound volume containing the structure of interest.

At 1710, the 3-D ultrasound volume is stored and/or employed (e.g., displayed, analyzed, utilized to show previously determined information, employ for an image guided procedure, etc.) as described herein and/or otherwise.

At least a portion of one or more of the methods discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method, comprising:
free hand rotating or free hand translating a first transducer array of a probe by rotating or translating the probe about or along a longitudinal axis of the probe through a plurality of angles or linear displacements in a cavity, wherein the rotating or the translating moves a first imaging plane of the first transducer array through an extent of a structure of interest;
transmitting ultrasound signals and receiving echo signals with the first transducer array concurrently with the rotating or the translating the first transducer array;
generating spatially sequential two-dimensional images of the structure of interest with the received echo signals for the plurality of the angles or the linear displacements;
identifying the plurality of the angles or the linear displacements based on the generated images and secondary information;
aligning the two-dimensional images based on the identified plurality of the angles or the linear displacements; and
combining the aligned two-dimensional images to construct a three-dimensional volume including at least the structure of interest.

2. The method of claim 1, wherein the secondary information includes a set of images for a transverse plane or sensed rotational or translational motion.

3. The method of claim 1, wherein the ultrasound probe is a biplane probe with the first transducer array and a second transducer array, which is transverse to the first transducer array, and further comprising:
transmitting second ultrasound signals and receiving second echo signals with the second transducer array with respect to a second imaging plane of the second transducer array concurrently with the rotating of the first transducer array;
generating a second image of the structure of interest with the received second echo signals; and
identifying the plurality of the angles for the spatially sequential two-dimensional images based on the rotation of the second imaging plane in the second image.

4. The method of claim 3, wherein the plurality of the angles is identified based on a fixed reference angle of the rotation of the second imaging plane.

5. The method of claim 3, wherein each angle of the plurality of the angles is identified based on an identified prior sequential angle of the second imaging plane.

6. The method of claim 1, further comprising, prior to the rotating or the translating of the first transducer array to generate the two-dimensional images:
translating the probe and transmitting third ultrasound signals and receiving third second echo signals with the second transducer array;
generating a third image of the structure of interest with the received third echo signals; and
positioning the second transducer array with respect to the structure of interest in the cavity based on the third image.

7. The method of claim 6, wherein positioning with the second transducer array includes placing the second transducer array at a center region of the structure of interest.

8. The method of claim 6, wherein positioning with the second transducer array includes:
placing the second transducer array at a first location at which the first imaging plane of the first transducer array covers a first sub-portion of the structure of interest for a first sweep of the first transducer array;
placing the second transducer array at a second different location at which the first imaging plane of the first transducer array covers a second different sub-portion of the structure of interest for a second sweep of the first transducer array; and
combining a first set of images from the first sweep with a second set of images from the second sweep to construct a single set of images covering both the first and the second sub-portions.

9. The method of claim 6, further comprising, prior to the rotating or the translating of the first transducer array to generate the two-dimensional images:
rotating the probe to place the first transducer array at a start angle at a first end of the structure of interest based on the third image.

10. The method of claim 9, further comprising:
rotating the probe to a stop angle at a second opposing end of the structure of interest based on the third image.

11. The method of claim 1, further comprising:
providing at least one of a visual or audible guide that indicates a predetermined rate of rotation or translation.

12. The method of claim 11, further comprising:
providing an indication of at least one of the predetermined rate of rotation or translation is satisfied or the predetermined rate of rotation or translation is not satisfied.

13. The method of claim 1, wherein the ultrasound probe includes at least one of an end-fire array or a sagittal array, and further comprising:
sensing the rotating with a rotation sensor of the probe; and
identifying the plurality of the angles based on an output of the rotation sensor, which is indicative of the rotation of the at least one of the end-fire or sagittal arrays.

14. The method of claim 1, wherein the ultrasound probe includes an axial array, and further comprising:
sensing the translating with a translation sensor of the probe; and
identifying the plurality of the displacements based on an output of the translation sensor, which is indicative of the displacement of the axial array.

15. An ultrasound probe, comprising:
at least one transducer array configured to transmit and receive echoes; and
a three-dimensional processor configured to align a set of image planes generated from the echoes for different free hand rotation angles of the at least one transducer array or different free hand displacements of the at least one transducer array based on a signal indicative of the different rotation angles or the different displacements, and configured to combine the aligned image planes to construct volumetric ultrasound image data of a structure of interest.

16. The ultrasound probe of claim 15, wherein the probe includes a biplane probe, and further comprising:
a second transducer array configured to transmit and receive echoes, wherein the second transducer array generates a reference image that indicates the different rotation angles, and wherein the three-dimensional processor determines the different rotation angles from the reference image and aligns the set of image planes based on the determined rotation angles.

17. The ultrasound probe of claim 15, wherein the probe includes an end-fire probe, and further comprising:
a sensor of the probe configured to sense a rotation of the probe, wherein the sensed rotation indicates the different rotation angles, and wherein the three-dimensional processor determines the different rotation angles from a signal from the sensor and aligns the set of image planes based on the signal.

18. The ultrasound probe of claim 15, wherein the probe includes a sagittal plane probe, and further comprising:
a sensor of the probe configured to sense a rotation of the probe, wherein the sensed rotation indicates the different rotation angles, and wherein the three-dimensional processor determines the different rotation angles from a signal from the sensor and aligns the set of image planes based on the signal.

19. The ultrasound probe of claim 15, wherein the probe includes an axial plane probe, and further comprising:
a sensor of the probe configured to sense a translation of the probe, wherein the sensed translation indicates the different displacements, and wherein the three-dimensional processor determines the different displacements from a signal from the sensor and aligns the set of image planes based on the signal.

20. A non-transitory computer readable medium encoded with computer executable instructions, which, when executed by a computer processor, causes the processor to:
acquire image planes with a rotating or translating first transducer array of a freehand rotating or translating probe;
determine rotation angles or displacements for the image planes based on one of an image of a transverse image plane or a signal from a motion sensor of the probe, wherein each image plane includes a different sub-portion of a structure of interest;
align the image planes based on the determined rotation angles or displacements; and
construct a three-dimensional data set of the structure of interest with the aligned image planes.
